(19) United States
(12) Patent Application Publication
(10) Pub. No.: US 2016/0237451 A1
(43) Pub. Date: Aug. 18, 2016

(54) CONFERRING RESISTANCE TO GEMINIVIRUSES IN PLANTS USING CRISPR/CAS SYSTEMS

(71) Applicant: REGENTS OF THE UNIVERSITY OF MINNESOTA, Minneapolis, MN (US)

(72) Inventors: Daniel F. Voytas, Falcon Heights, MN (US); Nicholas J. Baltes, Maple Grove, MN (US); Aaron W. Hummel, St. Louis, MO (US)

(21) Appl. No.: 15/025,690

(22) PCT Filed: Sep. 30, 2014
(86) PCT No.: PCT/US14/58188

§ 371 Date: Mar. 29, 2016

Related U.S. Application Data
(60) Provisional application No. 61/884,236, filed on Sep. 30, 2013.

(51) Int. Cl. C12N 15/82; C12N 15/11; C12N 9/22

(52) U.S. Cl. CPC C12N 15/8283; C12N 9/22; C12N 15/11; C12Y 301/00; C12N 2310/3513

(57) ABSTRACT

Materials and methods for conferring geminivirus resistance to plants, and particularly to materials and methods for using CRISPR/Cas systems to confer resistance to geminiviruses to plants.

### CROSS-REFERENCE TO RELATED APPLICATIONS

This application claims benefit of priority from U.S. Provisional Application Ser. No. 61/884,236, filed on Sep. 30, 2013.

### STATEMENT AS TO FEDERALLY SPONSORED RESEARCH

This invention was made with government support under DBI-0923827 and IOS-1339209, awarded by the National Science Foundation. The government has certain rights in the invention.

### TECHNICAL FIELD

This document relates to materials and methods for conferring geminivirus resistance to plants, and particularly to materials and methods for using CRISPR/Cas systems to confer resistance to geminiviruses to plants.

### BACKGROUND

Geminiviruses are a major plant pathogen, responsible for significant crop losses worldwide (Mansoor et al. Trends Plant Sci. 8:128-134, 2003). Due to a large host range, geminiviruses are capable of causing disease in a wide variety of plants, including species from both monocotyledonous (e.g., maize and wheat) and dicotyledonous (e.g., tomato and cassava) groups. Examples of geminiviruses include the cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, and tomato pseudo-curly top virus.

Methods for reducing geminivirus-related disease have been developed (see, e.g., Vanderschuren et al., Plant Biotechnol. J. 5:207-220, 2007), but efforts to generate geminivirus-resistant plants have been met with limited success, as there is a limited availability of geminivirus resistance genes from wild relatives or cultivars. In addition, plants that do have resistance are challenged by the frequently-evolving DNA virus, which is capable of changing its genome organization so as to overcome resistance. Genome engineering attempts to achieve geminivirus resistance typically have involved introducing into the plant genome a foreign DNA sequence encoding a product that directly interferes with the geminivirus life cycle. For example, geminivirus-resistant tomato plants were generated by introducing the gene coding for the geminivirus replication-associated protein into the plants. Before the plants could be commercialized, however, new geminiviruses emerged that were capable of causing disease in these plants (Moffat Science 286:1835, 1999).

### SUMMARY

The present document is based in part on the discovery of effective genome-engineering methods for increasing plant resistance to geminiviruses. The methods provided herein utilize the prokaryotic adaptive immune system known as the Clustered Regularly Interspersed Short Palindromic Repeats (CRISPR)/CRISPR-associated (Cas) system, which includes a nuclease known as Cas9. The methods can include, for example, (1) using a nuclease-active version of Cas9 to induce targeted double-strand breaks (DSBs) in the geminivirus double-stranded DNA replication intermediate, or single-strand breaks in the geminivirus single-stranded form, (2) using a nickase version of Cas9 to introduce DNA nicks in the geminivirus genome, or (3) using a nuclease-dead version of Cas9 to block the function or transcription of virus proteins. These methods also can be applied to symptom-modulating DNA satellites that are associated with geminiviruses. In addition, the CRISPR/Cas technology can be multiplexed, enabling the targeting of multiple different regions on the same geminivirus, or on multiple geminiviruses. The fact that CRISPR/Cas systems can be modified to function as endonucleases, nickases, or physical blockades may be useful for optimizing the system for use in specific plant species. For example, plants with large genomes may not tolerate constitutive expression of Cas endonucleases due to off-target DSBs. In such cases, the nickase or nuclease-dead versions of the Cas endonuclease may be particularly useful.

In one aspect, this document features a method for generating a plant cell having the potential for increased resistance to geminivirus infection. The method can include transforming a plant cell with (i) a first nucleic acid containing a sequence that encodes a Clustered Regularly Interspersed Short Palindromic Repeats-associated system (Cas) protein, and (ii) a second nucleic acid containing one or more Clustered Regularly Interspersed Short Palindromic Repeats (CRISPR) RNA (crRNA) sequences and a trans-activating crRNA (tracrRNA) sequence targeted to one or more geminivirus sequences, such that the sequence encoding the Cas protein, the one or more crRNA sequences, and the tracrRNA sequence are stably integrated into the genome of the plant cell. The method can further include maintaining the plant cell under conditions in which nucleic acids (i) and (ii) are expressed. The plant can be selected from the group consisting of tobacco, cabbage, wheat, miscanthus, potato, rice, squash, bean, beet, maize, spinach, cassava, pepper, cotton, tomato, and turnip. The Cas protein can be a Cas9 protein with nuclease activity, a Cas9 protein with nickase activity (e.g., a D10A or H840A nickase), or a Cas9 protein without nuclease activity (e.g., a D10A and H840A protein without nuclease activity). The sequence encoding the Cas protein can be operably linked to a constitutive promoter, an inducible promoter, or a plant tissue specific promoter. The crRNA and tracrRNA sequences can be targeted to a sequence contained within a geminivirus genome. The crRNA and tracrRNA sequences can be targeted to a sequence within a geminivirus genome that is conserved across two or more species of geminivirus. The crRNA and tracrRNA sequences can be targeted to one or more of the conserved replication-associated protein (Rep) binding sequence present within the geminivirus origin of replication, the conserved sequence present within the geminivirus Rep coding sequence, the stem-loop structure within the geminivirus origin of replication, the TAATATTAC sequence present in the apex of the geminivirus stem-loop structure, the nuclear shuttling protein coding sequence, and the movement protein coding sequence. The crRNA and tracrRNA sequences can be targeted to DNA beta molecules. The second nucleic acid can include one crRNA sequence, or more than one crRNA sequence. Each of the one or more crRNA sequences can be fused to a tracrRNA sequence. The tracrRNA sequence and the one or more crRNA sequences can be operably linked to a constitutive promoter (e.g., an RNA polymerase III promoter or an RNA polymerase II promoter), an inducible promoter, or a plant tissue specific promoter. The plant cell can be in a plant, and the transforming step can include Agrobacterium-mediated transformation, electroporation transformation, polyethylene glycol (PEG) transformation, or biolistic transformation. The first nucleic acid and the second nucleic acid can be in a single vector or in separate vectors. In some embodiments, the second nucleic acid can encode a polycistronic message containing a tracrRNA sequence and one or more crRNA sequences, or containing a cr/tracrRNA hybrid (gRNA) sequence. The method can further include transforming the plant cell with (iii) a third nucleic acid containing a sequence that encodes a protein for processing a transcript expressed from the second nucleic acid. The third nucleic acid can contain, for example, a sequence encoding a type III CRISPR/Cas-associated Csy4 protein. The sequence encoding the protein for processing the transcript can be operably linked to a constitutive promoter, an inducible promoter, or a plant tissue specific promoter.

In another aspect, this document features a plant, plant part, or plant cell that has increased resistance to geminivirus infection, where the genome of the plant, plant part, or plant cell contains a first nucleic acid encoding a Cas protein, and a second nucleic acid containing one or more crRNA sequences and a tracrRNA sequence targeted to one or more geminivirus sequences. The plant can be selected from the group consisting of tobacco, cabbage, wheat, miscanthus, potato, rice, squash, bean, beet, maize, spinach, cassava, pepper, cotton, tomato, and turnip. The Cas protein can be a Cas9 protein with nuclease activity, a Cas9 protein with nickase activity (e.g., a D10A or H840A nickase), or a Cas9 protein without nuclease activity (e.g., a D10A and H840A protein without nuclease activity). The sequence encoding the Cas protein can be operably linked to a constitutive promoter, an inducible promoter, or a plant tissue specific promoter. The crRNA and tracrRNA sequences can be targeted to a sequence within a geminivirus genome. The crRNA and tracrRNA sequences can be targeted to a sequence within a geminivirus genome that is conserved across two or more species of geminivirus. The crRNA and tracrRNA sequences can be targeted to one or more of the conserved Rep binding sequence present within the geminivirus origin of replication, the conserved sequence present within the geminivirus Rep coding sequence, the stem-loop structure within the geminivirus origin of replication, the TAATATTAC sequence present in the apex of the geminivirus stem-loop structure, the nuclear shuttling protein coding sequence, and the movement protein coding sequence. The crRNA and tracrRNA sequences can be targeted to DNA beta molecules. The second nucleic acid can contain one crRNA sequence or more than one crRNA sequence. Each of the one or more crRNA sequences can be fused to a tracrRNA sequence. The tracrRNA sequence and the one or more crRNA sequences can be operably linked to a constitutive promoter (e.g., an RNA polymerase III promoter or an RNA polymerase II promoter), an inducible promoter, or a plant tissue specific promoter. The second nucleic acid can encode a polycistronic message containing a tracrRNA sequence and one or more crRNA sequences, or containing a cr/tracrRNA hybrid (gRNA) sequence. The plant, plant part, or plant cell can further contain a third nucleic acid that contains a sequence encoding a protein for processing a transcript expressed from the second nucleic acid. The third nucleic acid can contains a sequence encoding a type III CRISPR/Cas-associated Csy4 protein. The sequence encoding the protein for processing the transcript can be operably linked to a constitutive promoter, an inducible promoter, or a plant tissue specific promoter.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a representative amino acid sequence for the Cas9 protein from Streptococcus pyogenes (SEQ ID NO:1). The aspartic acid (D) at position 10 and the histidine (H) at position 840 are circled. When D10 is mutated to an alanine (A) or when H840 is mutated to A, Cas9 loses nuclease activity from this domain and converts to a nickase. Mutation of H840 to A, combined with a D10A mutation, can abolish nuclease activity.

FIG. 2 is an illustration of a CRISPR/Cas system. Cas9 protein is guided to a target DNA sequence using an RNA molecule (crRNA and tracrRNA complex) with about 20 nucleotides of homologous sequence. A protospacer adjacent motif (PAM) sequence (NGG) immediately downstream of the target sequence is required for cleavage.

FIG. 3 is an illustration of a Cas9:gRNA complex. To generate the gRNA, a linker-loop sequence connects the 3′ nucleotide of the crRNA to the 5′ nucleotide of the tracrRNA.

FIG. 4 is an illustration of a mastrevirus genome and a bipartite begomovirus genome, with arrows indicating possible CRISPR/Cas target regions. SIR, small intergenic region; LIR, large intergenic region; 1, nonanucleotide sequence at the apex of the stem loop structure; 2, stemloop structure; 3, Rep binding domain; 4, Motif I in the Rep coding sequence; 5, Motif II in the Rep coding sequence; 6, Motif III in the Rep coding sequence; 7, Retinoblastoma binding domain; 8, oligo-binding sequence required for complementary strand synthesis; 9, V2 coding sequence; 10, V1 coding sequence; 11, AV1 coding sequence; 12, BV1 coding sequence; 13, BC1 coding sequence.

FIG. 5 is an illustration of the general organization of T-DNA vectors used in this study. Cas9 coding sequence from Streptococcus pyogenes (codon optimized for expression in plants) is placed downstream of a 2x35S promoter. Downstream of Cas9 is an Arabidopsis thaliana RNA pol III promoter (AtU6 or At7SL) followed by gRNA sequence. In cases where the CRISPR/Cas system is used to simultaneous target multiple sites, RNA pol III promoters and gRNAs are arranged in tandem. To reduce repetitive sequences, the RNA pol III promoters were alternated (e.g., At7SL:gRNA:AtU6:gRNA). Also present within the T-DNA is a NPTII antibiotic-resistance marker (not shown). NPTII is used for selecting transgenic cells and regenerating pants. Brackets and the letter n surrounding the gRNA refer to the ability of these vectors to harbor multiple gRNAs in tandem.

FIG. 6 illustrates an approach to test the effectiveness of CRISPR/Cas reagents against their target geminivirus. Two different Agrobacterium strains are mixed and infiltrated into Nicotiana benthamiana leaf tissue. One strain contains geminivirus vectors harboring GFP (upper right). The other strain contains CRISPR/Cas reagents (upper left).

FIG. 7 is an example of GFP expression in a leaf delivered geminivirus replicons, Cas9 and different gRNAs. A single leaf was infiltrated with several different samples of Agrobacterium and left on the plant for five days (left image). Five days post infiltration, GFP expression was captured and quantified using image analysis software (right).

FIG. 8 contains images of GFP expression in leaf tissue after delivery of control vectors (Replicon; Replicon+dCas9+TGMV gRNA; Replicon+Cas9+TGMV gRNA) and vectors containing gRNAs of interest (Replicon+Cas9+RBS (+) gRNA; Replicon+Cas9+MIII (+) gRNA; Replicon+Cas9+RBS (+) gRNA+MIII (+) gRNA). Each leaf was infiltrated with six different Agrobacterium samples. Images were taken five days post infiltration using the same exposure and magnification. dCas9, catalytically-dead Cas9; TGMV, tomato golden mosaic virus; RBS, rep binding sequence; MIII, motif III; (+), gRNA is complementary to the plus strand of the geminivirus; (−), gRNA is complementary to the minus strand of the geminivirus.

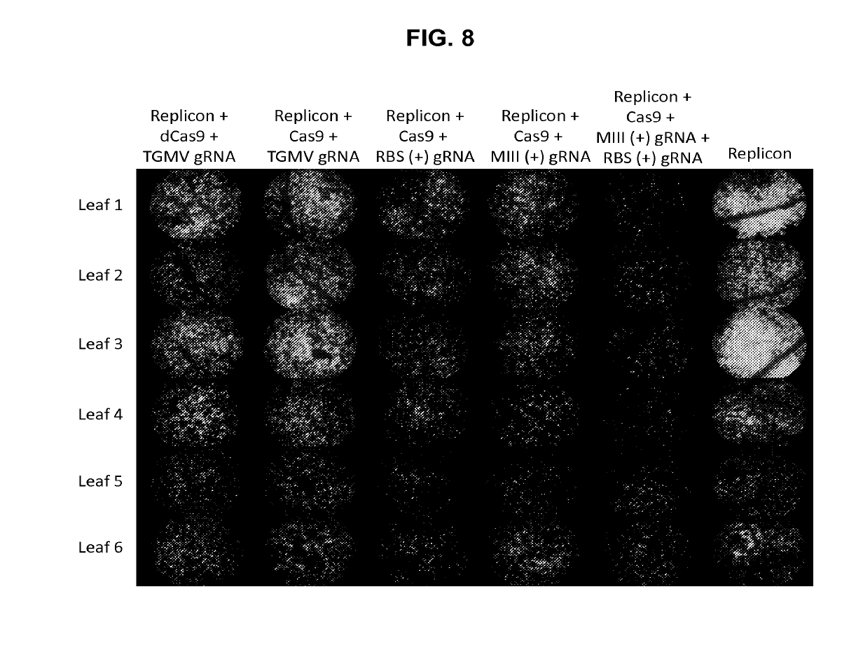

FIG. 9 is a graph showing the average GFP intensity from leaf tissue after delivery of control and experimental vectors as described in FIG. 7. The average GFP intensity was quantified using image analysis software and graphed. To reduce the variability in results due to age/health of the different infiltrated leaves, data were normalized in each leaf to a control (Replicon+Cas9+TGMV gRNA). Error bars represent the standard deviation from six leaves that came from six different plants.

FIG. 10 is a graph showing the average GFP intensity from leaf tissue after delivery of control vectors (Replicon+Cas9+TGMV gRNA) and vectors containing each of the gRNAs listed in TABLE 1, including a vector containing two gRNAs targeting RBS (+) and MIII (+). To reduce the variability in results due to age/health of the different infiltrated leaves, data were normalized in each leaf to the control (Replicon+Cas9+TGMV gRNA). Error bars represent standard deviation. N refers to the total number of leaves from different plants that were infiltrated with the corresponding vectors. Dark gray bars indicate that data from two independent experiments was combined. Light gray bars represent data from one experiment.

FIG. 11 is a graph showing the average colony forming units (CFUs) obtained from tissue treated with replicon only or Replicon+RBS gRNA+MIII gRNA. Total DNA was extracted from 0.5 cm leaf punches harvested 5 days after infiltration. DNA was treated with DpnI to remove contaminating T-DNA vector and Replicons derived from prokaryotic cells. DpnI-treated total DNA was transformed into high efficiency E. coli and plated on Luria-Bertani agar containing 50 μg/mL carbenicillin and incubated overnight at 37° C. before colonies were counted. Values are normalized to Replicon only. Capped bars represent standard deviation. Replicon only and Replicon+RBS gRNA+MIII gRNA values are significantly different (p=0.0017).

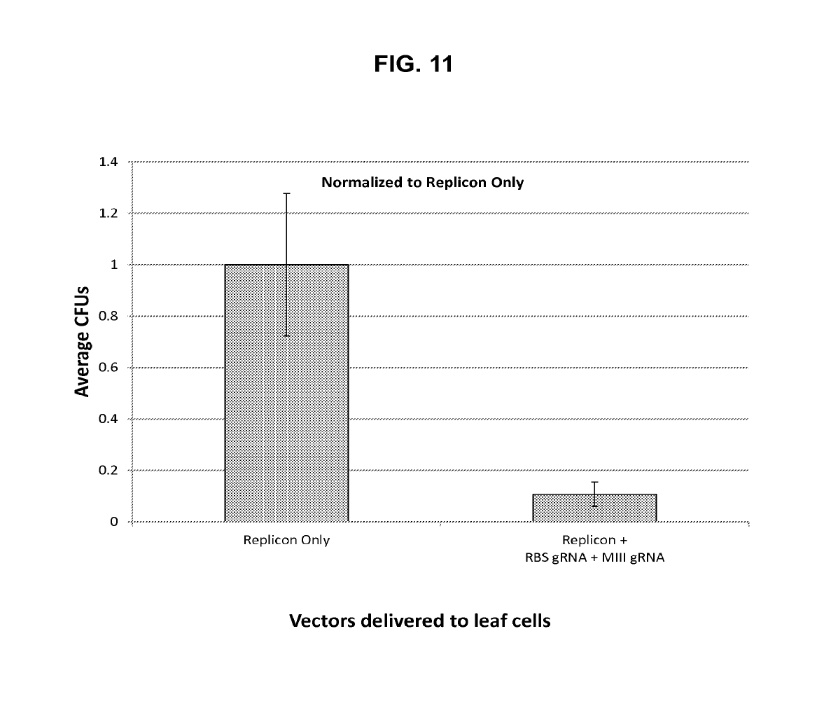

FIG. 12 is an illustration of CRISPR/Cas T-DNA vectors used to make transgenic N. benthamiana plants.

FIG. 13 is an illustration of CRISPR/Cas T-DNA vectors used to make transgenic Solanum lycopersicum plants.

### DETAILED DESCRIPTION

The methods described herein can be used for engineering plants to have pre-programmed CRISPR/Cas systems that target geminivirus DNA sequences. In its native context, the CRISPR/Cas system provides bacteria and archaea with immunity to invading foreign nucleic acids (Jinek et al. Science 337:816-821, 2012). The CRISPR/Cas system is functionally analogous to eukaryotic RNA interference, using RNA base pairing to direct DNA or RNA cleavage. This process relies on (a) small RNAs that base-pair with sequences carried by invading nucleic acid, and (b) a specialized class of Cas endonucleases that cleave nucleic acids complementary to the small RNA. The CRISPR/Cas system can be reprogrammed to create targeted double-strand DNA breaks in higher-eukaryotic genomes, including animal and plant cells (Mali et al. Science 339:823-826, 2013; and Li et al. Nature Biotechnology 31(8): 688-691, 2013). Further, by modifying specific amino acids in the Cas protein that are responsible for DNA cleavage, the CRISPR/Cas system can function as a DNA nickase (Jinek et al., supra), or as a DNA binding protein that has no nuclease or nickase activity but is capable of interfering with incoming proteins, including RNA polymerases (Qi et al. Cell 152:1173-1183, 2013).

Directing DNA DSBs, single strand nicks, or binding of the Cas9 protein to a particular sequence requires crRNA and tracrRNA sequences that aid in directing the Cas/RNA complex to target DNA sequence (Makarova et al., Nat Rev Microbiol, 9(6):467-477, 2011). The modification of a single targeting RNA can be sufficient to alter the nucleotide target of a Cas protein. In some cases, crRNA and tracrRNA can be engineered as a single cr/tracrRNA hybrid to direct Cas activity (also referred to herein as a “guide RNA” (gRNA)), whether as a nuclease, a nickase, or a DNA binding protein.

This document provides methods for using CRISPR/Cas systems to generate plants, plant tissues, plant parts, and plant cells that have increased resistance to geminivirus. The term “increased resistance,” as used herein, means that a plant, plant part, or plant cell is less severely affected by geminivirus infection than a corresponding plant, plant part, or plant cell that does not contain CRISPR/Cas components as described herein. For example, a plant with increased resistance to geminivirus will display fewer or milder symptoms (e.g., leaf curling, chlorotic lesions, and stunting) when exposed to geminivirus, as compared to a corresponding plant that does not have increased geminivirus resistance. In some cases, symptoms of geminivirus infection can be scored (e.g., using a scale with no observable symptoms at one end and severe symptoms at the other). In such cases, the difference between the score for a plant with increased geminivirus resistance and the score for no observable symptoms will be less than the difference between the score for a corresponding plant without increased geminivirus resistance and the score for no observable symptoms.

The methods can include, for example, transforming a plant, plant part (e.g., a leaf, stem, or root, or a portion thereof), or a plant cell (e.g., a leaf cell, stem cell, root cell, or protoplast) with (i) a first nucleic acid encoding a Cas protein, and (ii) a second nucleic acid containing one or more crRNA sequences and one or more tracrRNA sequences that are targeted to one or more geminivirus sequences, such that nucleic acids (i) and (ii) are stably integrated into the genome of the plant, plant part, or plant cell. The methods also can include maintaining the plant, plant part, or plant cell under conditions in which nucleic acids (i) and (ii) are expressed.

The methods provided herein can be useful for any type of crop or economically valuable plant that is susceptible to geminivirus infection and is amenable to stable DNA integration. For example, the methods provided herein can be useful for, without limitation, grasses and members of the cereal, vegetable, and fiber crops, such as tobacco, cabbage, wheat, miscanthus, potato, rice, squash, bean, beet, maize, spinach, cassava, pepper, cotton, tomato, and turnip.

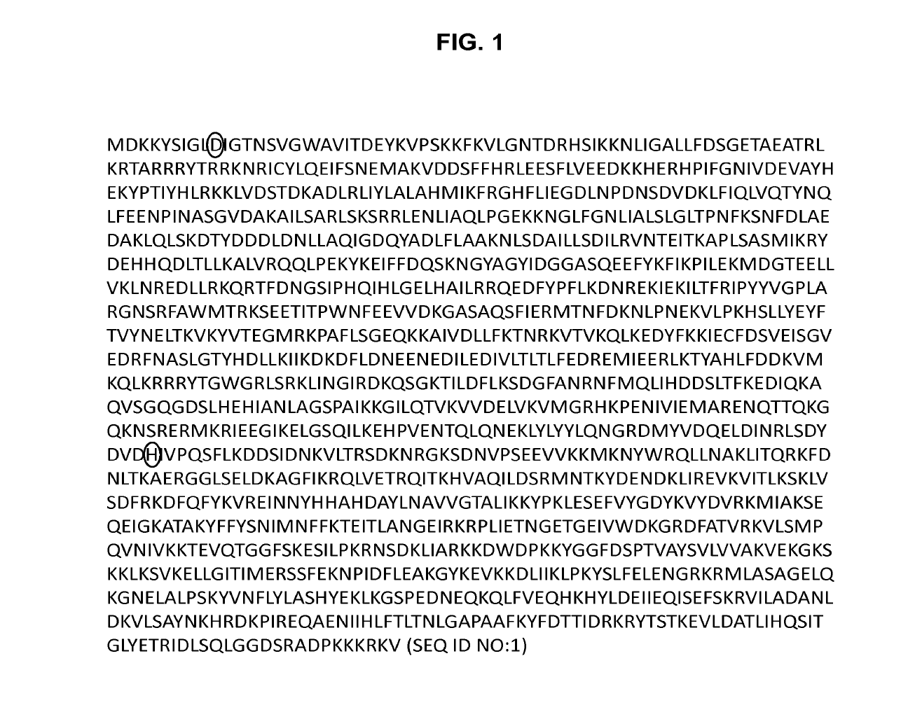

In some embodiments, the first nucleic acid can encode a Cas protein that has nuclease activity and can generate a DSB at a preselected target sequence when complexed with crRNA and tracrRNA or gRNA. For example, the first nucleic acid can encode a wild type Cas9 protein, or a Cas9 protein that contains one or more mutations (e.g., substitutions, deletions, or additions) within its amino acid sequence as compared to the amino acid sequence of a corresponding wild type Cas9 protein, where the mutant Cas9 retains nuclease activity. In some embodiments, additional amino acids may be added to the N- and/or C-termini. For example, Cas9 protein can be modified by the addition of a VP64 activation domain or a green fluorescent protein to the C-terminus, or by the addition of nuclear-localization signals to both the N- and C-termini (see, e.g., Mali et al. Nature Biotechnology 31:833-838, 2013; and Cong et al. Science 339:819-823). A representative Cas9 amino acid sequence is shown in FIG. 1.

In some embodiments, the first nucleic acid can encode a Cas protein that does not have nuclease activity (i.e., that cannot generate DSBs within a target sequence), but has nickase activity and can generate one or more single strand nicks within a preselected target sequence when complexed with crRNA and tracrRNA. For example, the first nucleic acid can encode a Cas9 D10A nickase protein in which an alanine residue is substituted for the aspartic acid at position 10, or a Cas9 H840A protein in which an alanine residue is substituted for the histidine at position 840.

In some embodiments, the first nucleic acid can encode a “nuclease-dead” Cas protein that has neither nuclease nor nickase activity, but can bind to a preselected target sequence when complexed with crRNA and tracrRNA. Such Cas proteins can interfere with the activity of other proteins that may act at or near the preselected target sequence, including RNA polymerases. For example, the first nucleic acid can encode a D10A H840A Cas9 protein in which alanine residues are substituted for the aspartic acid at position 10 and the histidine at position 840, or a D10A D839A H840A N863A Cas9 protein in which alanine residues are substituted for the aspartic acid residues at positions 10 and 839, the histidine residue at position 840, and the asparagine residue at position 863. See, e.g., Mali et al., Nature Biotechnology, supra.

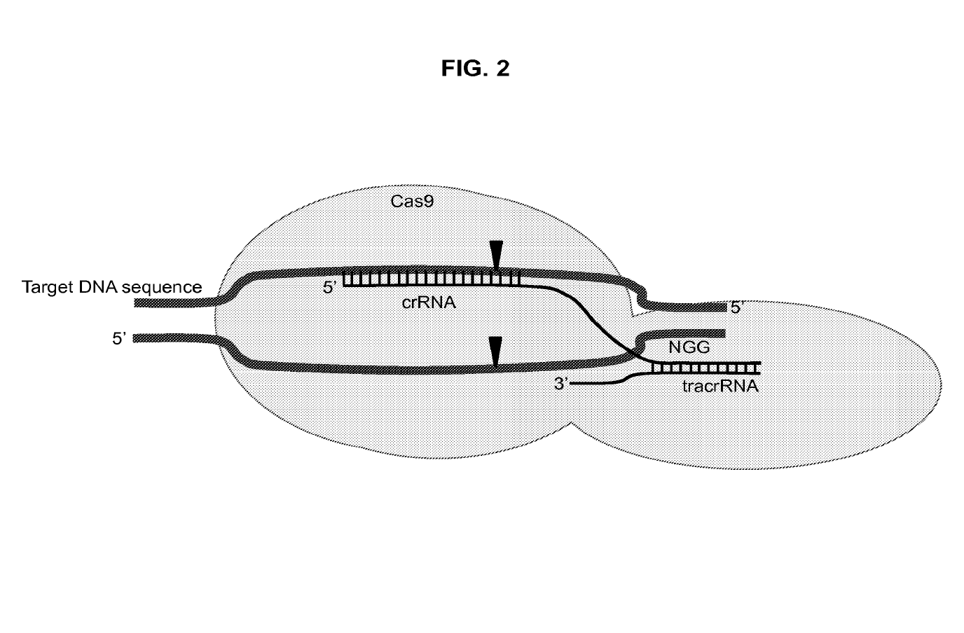

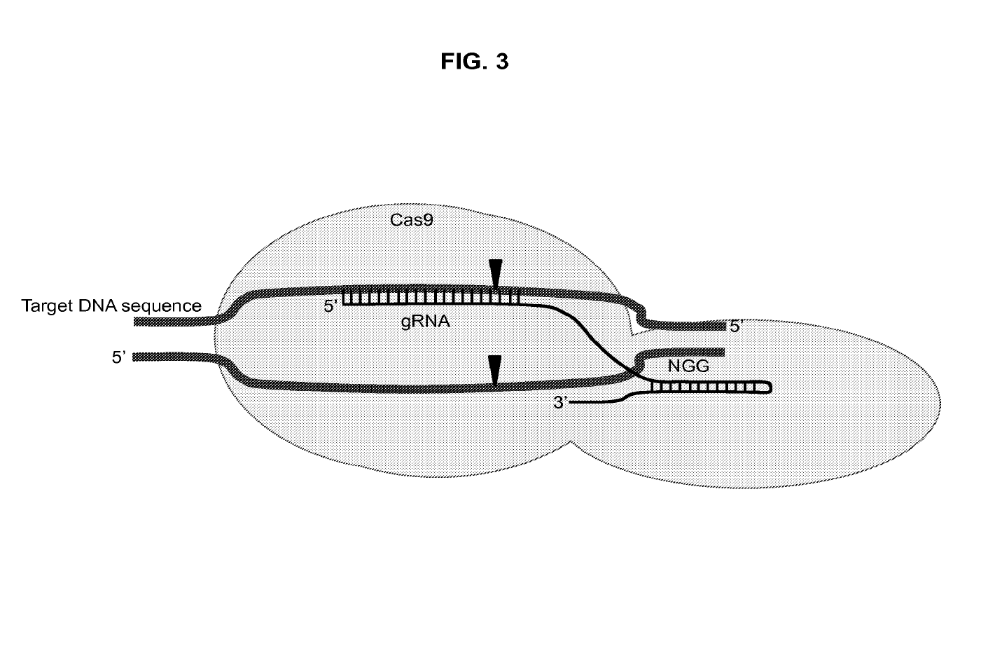

The second nucleic acid can contain one or more (e.g., one, two, three, four, five, or more than five) crRNA sequences, and one or more (e.g., one, two, three, four, five, or more than five) tracrRNA sequences, where the crRNA sequences are targeted to one or more (e.g., one, two, three, four, five, or more than five) geminivirus sequences. For example, each of the one or more crRNA sequences can contain a region that is homologous to a geminivirus sequence, such that the one or more crRNA sequences are targeted to different geminivirus sequences. The tracrRNA hybridizes with the crRNA, and together they guide the Cas protein to the target sequence (FIG. 2). In some embodiments, when multiple crRNA sequences are used, each crRNA sequence can contain a different geminivirus homology region but the same tracrRNA hybridizing region. Thus, in such embodiments, the second nucleic acid can contain more than one crRNA sequence but a single tracrRNA sequence. Further, in some embodiments, the crRNA and tracrRNA sequences can be artificially fused into cr/tracrRNA hybrid (gRNA) sequences, as depicted in FIG. 3.

The first nucleic acid and the second nucleic acid can be included within a single nucleic acid construct, or in separate constructs. Thus, while in some cases it may be most efficient to include the sequences encoding the Cas protein, the crRNA(s), and the tracrRNA(s) in a single construct (e.g., a single vector), in some embodiments, the crRNA and tracrRNA sequences can be present in separate nucleic acid constructs (e.g., separate vectors). As used herein, a “vector” is a replicon, such as a plasmid, phage, or cosmid, into which another DNA segment may be inserted so as to bring about the replication of the inserted segment. For example, if a geminivirus is to be targeted at five different sequences, seven different nucleic acid constructs could be used for integration into the host genome (e.g., a first nucleic acid encoding the Cas protein, a second nucleic acid encoding the tracrRNA, and third through seventh nucleic acids encoding the crRNAs).

The geminivirus homology regions within each crRNA sequence can be between about 10 and about 40 (e.g., 10, 11, 12, 13,14, 15, 16, 17, 18, 19, 20, 21, 22, 23, 24, 25, 26, 27, 28, 29, 30, 31, 32, 33, 34, 35, 36, 37, 38, 39, or 40) nucleotides in length. The tracrRNA hybridizing region within each crRNA sequence can be between about 8 and about 20 (e.g., 8, 9, 10, 11, 12, 13, 14, 15, 16, 17, 18, 19, or 20) nucleotides in length. The overall length of a crRNA sequence can be, for example, between about 20 and about 80 (e.g., 20, 25, 30, 35, 40, 45, 50, 55, 60, 65, 70, 75, or 80) nucleotides, while the overall length of a tracrRNA can be, for example, between about 10 and about 30 (e.g., 10, 12, 14, 16, 18, 20, 22, 24, 26, 28, or 30) nucleotides. The overall length of a gRNA sequence, which includes a geminivirus homology region and a stem loop region that contains a crRNA/tracrRNA hybridizing region and a linker-loop sequence, can be between about 30 and about 110 (e.g., 30, 35, 40, 45, 50, 55, 60, 65, 70, 75, 80, 85, 90, 95, 100, 105, 110, 115, 120, 125, or 130) nucleotides.

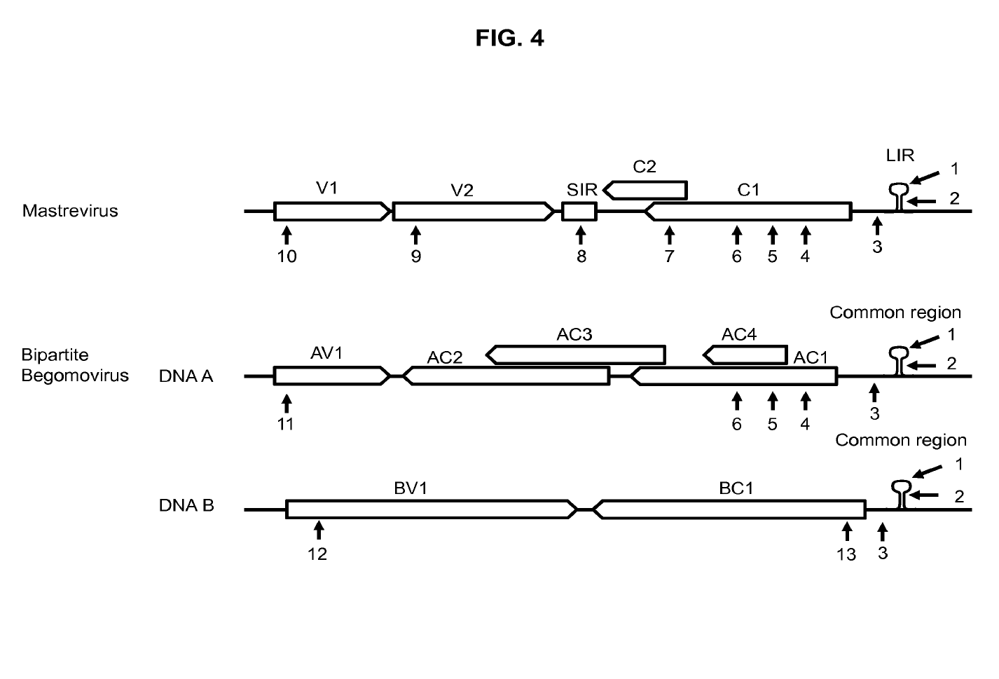

Any geminivirus sequence can be targeted. In some embodiments, it can be useful to target one or more geminivirus sequences that are conserved across two or more species of geminivirus (which include, for example, tomato golden mosaic virus (TGMV), bean yellow dwarf virus (BeYDV), tomato yellow leaf curl virus (TYLCV), cabbage leaf curl virus, wheat dwarf virus, tomato leaf curl virus, maize streak virus, tobacco leaf curl virus, beet curly top virus, spinach severe curly top virus, bean golden mosaic virus, tomato pseudo-curly top virus, and turnip curly top virus). Examples of geminivirus sequences that can be targeted by crRNA sequences in the methods provided herein can include, without limitation, the conserved replication-associated protein (Rep) binding sequence within the origin of replication (e.g., TGMV GGTAGTAAGGTAG (SEQ ID NO:2), or mild strain of bean yellow dwarf virus (BeYDV-m) TGGAGGCATGGAGGCA (SEQ ID NO:3)), the conserved sequence present within the Rep coding sequence (e.g., BeYDV-m TTCCTTACCTAT (Motif I, SEQ ID NO:4), BeYDV-m CACTATCATGCTCTTCTC CAG (Motif II, SEQ ID NO:5), BeYDV-m GTCCTTGATTACATATCAAAG (Motif III, SEQ ID NO:6), BeYDV-m CTTCGCTGCCACGAA (retinoblastoma (Rb) binding domain, SEQ ID NO:7), TGMV TTTCTTACATATCCTCAGTGC (Motif I, SEQ ID NO:21), TGMV CACCTCCACGTGCTTATTCAG (Motif II, SEQ ID NO:22), or TGMV GTCAAGACGTACATCGACAAA (Motif III, SEQ ID NO:23)), the stem-loop structure within the origin of replication (e.g., BeYDV-m GCGACAAGGGGGGGCCCACGCCG (SEQ ID NO:8)), the TAATATTAC nonanucleotide sequence present in the apex of the stem-loop structure, the nuclear shuttling protein (NSP) coding sequence (e.g., cabbage leaf curl virus ATGTATCCTACAAAGTTTAGGCGTGG (SEQ ID NO:9)), and/or the movement protein (MP) coding sequence (e.g., cabbage leaf curl virus CTATGTAATTAAACGCATTTGGAG (SEQ ID NO:10)). Locations of possible CRISPR/Cas targets also are depicted in FIG. 4, which provides a diagram of the mastrevirus and begomovirus genomes and indicates possible target sites with arrows.

Further, since geminiviruses are circular, single-stranded (plus strand) DNA molecules that replicate through double-stranded DNA intermediates, the geminivirus target sequence (i.e., the geminivirus DNA that base-pairs with crRNA) can be present on the plus or minus strand. Examples of target sequence present on the plus strand include, without limitation, GGTAGTAAGGTAG (the conserved Rep binding sequence within the origin of replication of TGMV, SEQ ID NO:2), TGGAGGCATGGAGGCA (the conserved Rep binding sequence within the origin of replication of BeYDV-m, SEQ ID NO:3), ATAGGTAAGGAA (Motif I within the Rep coding sequence of BeYDV-m, SEQ ID NO:11), CTGGAGAAGAGCATGATAGTG (Motif II within the Rep coding sequence of BeYDV-m, SEQ ID NO:12), CTTTGATATGTAATCAAGGAC (Motif III within the Rep coding sequence of BeYDV-m, SEQ ID NO:13), TTCGTGGCAGCGAAG (Rb binding domain within the Rep coding sequence of BeYDV-m, SEQ ID NO:14), GCGACAAGGGGGGGCCCACGCCG (the stem-loop structure within the origin of replication of BeYDV-m, SEQ ID NO:8), TAATATTAC (the nonanucleotide sequence present in the apex of the stem-loop structure of BeYDV-m), ATGTATCCTACAAAGTT TAGGCGTGG (the NSP coding sequence of the cabbage leaf curl virus; SEQ ID NO:9), and CTATGTAATTAAACGCATTT (the MP coding sequence of the cabbage leaf curl virus; SEQ ID NO:15). Examples of target sequence present on the minus strand include, without limitation, CTACCTTACTACC (the conserved Rep binding sequence within the origin of replication of TGMV, SEQ ID NO:16), TGCCTCCATGCCTCCA (the conserved Rep binding sequence within the origin of replication of BeYDV-m, SEQ ID NO:17), TTCCTTACCTAT (Motif I within the Rep coding sequence of BeYDV-m, SEQ ID NO:4), CACTATCATGCTCTTCTCCAG (Motif II within the Rep coding sequence of BeYDV-m, SEQ ID NO:5), GTCCTTGATTACATATCAAAG (Motif III within the Rep coding sequence of BeYDV-m, SEQ ID NO:6), CTTCGCTGCCACGAA (Rb binding domain within the Rep coding sequence of BeYDV-m, SEQ ID NO:7), CGGCGTGGGCCCCCCCTTGTCGC (the stem-loop structure within the origin of replication of BeYDV-m, SEQ ID NO:18), GTAATATTA (the nonanucleotide sequence present in the apex of the stem-loop structure of the BeYDV-m), CCACGCCTAAACTTT GTAGGATACAT (the NSP coding sequence of cabbage leaf curl virus, SEQ ID NO:19), and AAATGCGTTTAATTACATAG (the MP coding sequence of cabbage leaf curl virus, SEQ ID NO:20). Targeting either the plus strand or the minus strand can be beneficial.

In some embodiments, the crRNA and tracrRNA sequences can be targeted to one or more symptom-modulating DNA satellites (also referred to as betasatellites or DNA beta molecules) that commonly are associated with geminivirus infection. DNA beta molecules depend on the geminivirus for replication and spread within and between hosts. Most DNA beta molecules encode a protein, βC1, which acts as a pathogenicity factor to enhance viral replication and movement, and can contribute to disease symptoms. An example of a DNA beta molecule is the cotton leaf curl virus beta (5′-ACCGTGGGCGAGCGGTGTCTTTGGCGTTCCATGTGGGTCCCACAATATCCA AAAGAAGAATAATGGACTGGGTCAATGCAATTGGGCCTTAAATGAAATGGG CTTGGACCAGTAGATTCGAGACTGGGCCAATAGAATAAAACAACAAATGG ACTCATAATCAAAACAAAGTGTTTATTCATGTCAAATACATTACACACTCAC ACACACACAGTCGTACACACATCATATTCATCCCCTATACGTATATCAACTAA TGGGGCCTCATGCATCATCATTATATCAATAGCCTCTACCATGTCCTCCTGGC GAAAGTCCCGAACATGACAATCCCTATACATGATCTTTAATATATTATGTATC CCTTCCTCCAAATTGTTGAAGTCGAAAGGCGGTATGATCCCATCATGGCCGT ATGGGATCATGAAGGTCTTCTTTGCCAGGGCAGGTGATCTTGTTGAGCACA ATTCAATCCGCACAAGGATTGAATTGTCCTCGTGGATCTTCACGTCGACGGT AAATACCATCCCCTTCTCGTTAGTATACTTGATTGTCATTTGCATTTAATTATG AACAACACATGAGATGAATCTTCTTAAATAGCGTCCATATATTTGGATATATG GACATAATGCATATACGTGGTGCAATAATTATCATATGAATATGAGTGGAGAC ATATATGATTGATATCTACAGGACTATCAATCATCGTCAAGAAAAAAGGGAT AAAAGAAACCTTATTCATGAAAGGATTAGGTAGGGAAAAATAAAAGAAGG AAATTAAGGAAAAGAAAACCCACAATGAAATAATTAAGAAAAAAAAAAGA AAAAAAGAACACAAGAAAGGAAAACAGAAACATGAGCACAAACAGAAA CCTCTTGAGAAAATATTGGAGCGCAGCGGGAAAACCAAGAAAAACAAACC AAGGAAGACACTTGAGCAAAAAGGGAAAACACAAACTAAACTAGGAGGG TCCAACATGAAATTATGAAAAGTCCGTACACAGTAATTAATCATTAATTACT GCGCAGTAAATGTGAATAAAATTAACCCGAAGGGTTAATTTTCGAGACCCC GATAGGTAATTGAGTCCCCAATATATCGGGGACTCAATCGGGGTCATGAGA GAGAAATAAATCCCGGATACCGAAACTACCCTCAAAGCTGTGTCTGGAAGG CGCGTGGGAGTGCGCTGAAAAAGGTGACCTTCTCTCTCCAAAAACTCACC GGAACGGCCAAACTGGCTGATTCCGGCATCTAATCACGACACGCGCGGCG GTGTGTACCCCTGGGAGGGTAGACCACTACGCTACGCAGCAGCCTTAGCTA CGCCGGAGCTTAGCTCGCCCACGTTCTAATATT; SEQ ID NO:55)

Suitable target sequences typically are followed by a protospacer adjacent motif (PAM) sequence that is required for cleavage. The PAM sequence can be immediately downstream of the target sequence. As indicated in FIGS. 2 and 3, for example, an NGG PAM sequence downstream of the target sequence can be required for cleavage by a Cas9 nuclease (e.g., by a S. pyogenes Cas9). Alternatively, a NNNNGMTT PAM sequence can be required for cleavage by a Neisseria meningitides Cas9 protein.

In some embodiments, a plant, plant part, or plant cell also can be transformed with a third nucleic acid, which can contain a sequence encoding a protein for processing the tracrRNA transcripts and the one or more crRNA transcripts, or two or more gRNA transcripts, into separate molecules (e.g., when the tracrRNA and crRNA sequences or multiple gRNA sequences are expressed within a polycistronic message). The third nucleic acid can contain, for example, a sequence encoding a type III CRISPR/Cas-associated Csy4 protein.

The Cas coding sequence, the crRNA and tracrRNA (or gRNA) sequences, and (when included) the sequence encoding the protein for separating the tracrRNA and crRNA sequences can be independently and operably linked to promoters that are inducible, constitutive, cell specific, or tissue (e.g., plant tissue) specific (such as an egg apparatus-specific enhancer (EASE), cruciferin, napin, or rubisco small subunit promoter), or to promoters that are activated by alternative splicing of a suicide exon. Exemplary constitutive promoters include, without limitation, constitutive RNA pol II promoters such as the 35S, Nos-P, and ubiquitin promoters, and constitutive RNA pol III promoters such as the U6 promoter. Examples of inducible promoters include, without limitation, the virion-sense promoter from geminivirus, and the XVE promoter. In some embodiments, for example, a Cas coding sequence can be operably linked to an inducible XVE promoter, which can be activated by estradiol. Expression of the Cas protein in a plant can be activated by treating the plant with estradiol, and the expressed protein then can cleave, nick, or bind to geminivirus DNA at the target sequence—provided that crRNA and tracrRNA (or gRNA) sequences also are present and expressed.

In some embodiments, each gRNA (or crRNA and tracrRNA) can have its own promoter. In other embodiments, a polycistronic approach can be used to express multiple RNAs from one promoter. For example, multiple crRNAs can be expressed from a single promoter, along the lines of the bacterial pre-crRNA molecule, while the tracrRNA can be expressed from a separate promoter. In some cases, a polycistronic message can include one or more crRNA sequences and one or more tracrRNA sequences, or two or gRNA sequences.

The nucleic acid construct(s) containing the crRNA, tracrRNA, and Cas coding sequences can be stably integrated in the genome of whole plants by biolistic bombardment or by Agrobacterium mediated transformation. Alternatively, the system components can be delivered to a plant, plant tissue, plant part, or plant cell using Agrobacterium-mediated transformation, electroporation, polyethylene glycol (PEG) transformation, insect vectors, grafting, or DNA abrasion, according to methods that are standard in the art, including those described herein. In some embodiments, the system components can be delivered in a viral vector (e.g., a vector from a DNA virus such as, without limitation, geminiviruses (e.g., cabbage leaf curl virus, bean yellow dwarf virus, wheat dwarf virus, tomato leaf curl virus, maize streak virus, tobacco leaf curl virus, or TGMV), nanoviruses (e.g., Faba bean necrotic yellow virus), or a vector from an RNA virus such as, without limitation, a tobravirus (e.g., tobacco rattle virus or tobacco mosaic virus), a potexvirus (e.g., potato virus X), or a hordeivirus (e.g., barley stripe mosaic virus).

After a plant or plant cell is infected or transfected with nucleic acids encoding the Cas protein and the crRNA and tracrRNA sequences, any suitable method can be used to determine whether the CRISPR/Cas sequences have integrated into the genome of the plant or plant cell. For example, thermal asymmetric interlaced polymerase chain reaction (PCR) or Southern blotting of genomic DNA from a potentially transgenic plant, plant part, or plant cell, or from progeny thereof, can be used to assess whether integration has occurred.

In addition, any suitable method can be used to determine whether CRISPR/Cas sequences are expressed in a transgenic plant, plant portion, or plant cell. In some embodiments, for example, western blotting of cellular extracts can be used to determine whether the Cas protein is present, and Northern blotting of cellular RNA can be used to determine whether the crRNA and tracrRNA are expressed.

After it has been determined that a transgenic plant, plant part, or plant cell expresses the CRISPR/Cas components, any suitable method(s) can be used to propagate the plant, plant part, or plant cell to generate a population of transgenic plants that express the CRISPR/Cas components and thus have increased geminivirus resistance. Such methods include those that are standard in the art.

In addition to the methods described herein, this document also provides plants, plant parts, and plant cells that contain CRISPR/Cas components as described herein, and thus, when the CRISPR/Cas components are expressed, have increased geminivirus resistance.

The invention will be further described in the following examples, which do not limit the scope of the invention described in the claims.

### EXAMPLES

### Example 1

### Molecular Reagents for Achieving Resistance to Geminiviruses

To generate plants with CRISPR/Cas reagents capable of targeting geminiviruses, plasmid DNA (either transfer DNA (T-DNA) plasmid or conventional plasmid) is modified to encode a bacterial, human, or plant codon optimized Cas9 gene and crRNA and tracrRNA, or alternatively, a gRNA(s) (a synthetic fusion of the crRNA and tracrRNA). Transcription of Cas9 is controlled by a constitutive RNA pol II promoter (e.g., 35S, Nos-P, or ubiquitin promoter sequence) or an inducible promoter/system (e.g., the virion-sense promoter from geminiviruses, or an in planta activation vector (see, for example, Dugdale et al. The Plant Cell 25:2429-43, 2013). The Cas9 coding sequence is either a nuclease-active (WT) sequence, a nickase sequence (e.g., D10A), or the nuclease-dead sequence (D10A and H840A) (Jinek et al., supra; and Qi et al., supra). See, FIG. 1 for a representative amino acid sequence for the Cas9 protein from Streptococcus pyogenes (SEQ ID NO:1). Transcription of the gRNA is controlled either by a constitutive RNA pol III promoter (e.g., U6, U3, 7SL) or by a constitutive RNA pol II promoter (e.g., 35S, Nos-P, Ubq1, Ubq10), or by an inducible promoter (e.g., XVE; Zuo et al., 2000 Plant J 24:265-273). To direct the Cas9-RNA complex to geminivirus DNA, the crRNA or gRNA is modified to include 20 nucleotides that are complementary to sequence present in the geminivirus genome. Target sequences of interest may include the conserved Rep binding sequence (present within the origin of replication), the nucleotides within Rep coding sequence that specify conserved amino acids (e.g., Motif I, II and III, which are required for the initiation of rolling circle replication), the stem-loop structure within the origin of replication, or the conserved nonanucleotide sequence (TAATATTAC) that is present in the apex of the stem-loop structure. A list of CRISPR/Cas target sequences used in this study is found in TABLE 1. Target sequences were chosen because they are conserved at the nucleotide, amino acid, or secondary structure level within diverse geminivirus genomes.

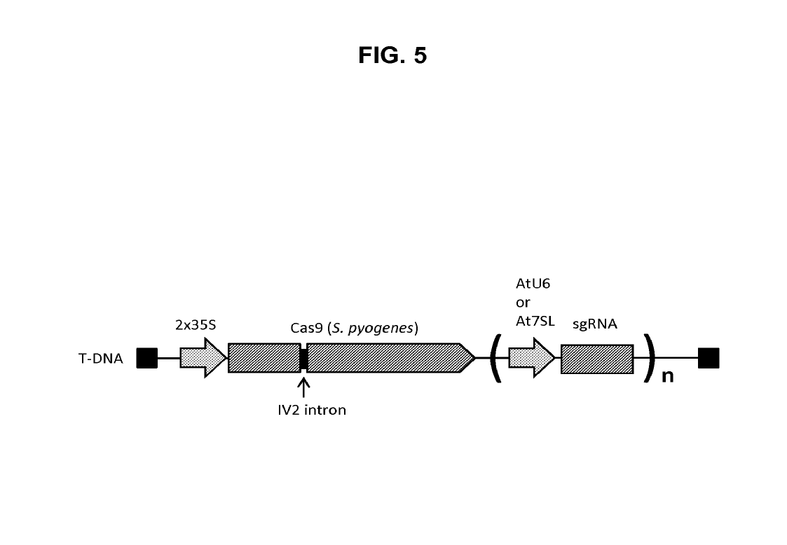

All CRISPR/Cas plasmids described in these examples were constructed using a pCAMBIA destination T-DNA plasmid (pCGS710). Within the T-DNA borders of pCGS710 were gateway recombination sites (2X35S:attR1:ccdbR:attR2) followed by a kanamycin-resistance marker. To enable facile cloning into pCGS710, Cas9 and gRNA(s) were cloned into multisite gateway entry vectors, pNJB91 and pNJB80, respectively, and as described elsewhere (Baltes et al., Plant Cell 1:151-163, 2014). To generate pNJB91 with Cas9, Cas9 coding sequence was PCR amplified from vectors described by Li et al. (Nature Biotechnol 31:688-691, 2013) using primers 5′-CTCCGAATTCGCCCTTCACC ATGGATTACAAGGATGATGATG (SEQ ID NO:24) and 5′-AAATGTTTGAACGAT CGGACGTCTCACTTCTTCTTCTTAGCCTG (SEQ ID NO:25). The resulting PCR product was digested with NcoI and AatII and cloned into pNJB91, generating pNJB184. To generate pNJB80 harboring gRNA sequence, gBlocks (IDT) were synthesized containing nucleotide sequences for the AtU6 promoter (Wang et al., RNA 14:903-13, 2008) followed by gRNA sequence (gNNNNNNNNNNNNNNNNNNNNgttttagagctaga aatagcaagttaaaataaggctagtccgttatcaacttgaaaaagtggcaccgagtcggtgcttttttt; SEQ ID NO:26); the lower case letters indicate constant gRNA sequence; the uppercase letters represent the 20 nt sequence that is responsible for directing Cas9 cleavage. To enable cloning of oligonucleotides into the 5′ region of the gRNA, two inverted Type IIS restriction enzyme sites (Esp31) were positioned downstream of the RNA Pol III promoter. The resulting gBlock was cloned into pNJB80, generating pPAA033. All BeYDV target sequences listed in TABLE1 were synthesized as oligos (containing matching overhangs with an Esp31 digested pPAA033 plasmid) and cloned into pPAA033. To generate T-DNA vectors containing Cas9 and gRNA sequence, a multisite gateway reaction was performed using pCGS710, pNJB184, and pPAA033 (containing BeYDV target sequences). The resulting T-DNA plasmids (FIG. 5) are hereafter labeled with descriptive terms; for example, Cas9+RBS (+) gRNA refers to a T-DNA plasmid containing 2x35S:Cas9 followed by AtU6 and a gRNA designed to be complementary to plus strand Rep binding sequence.

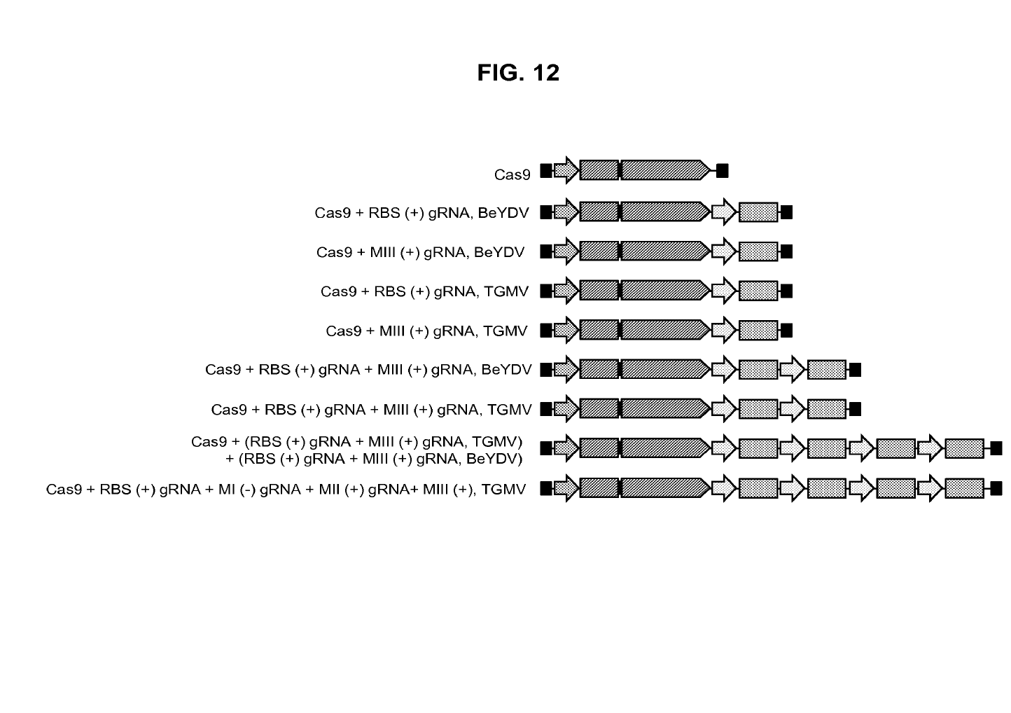

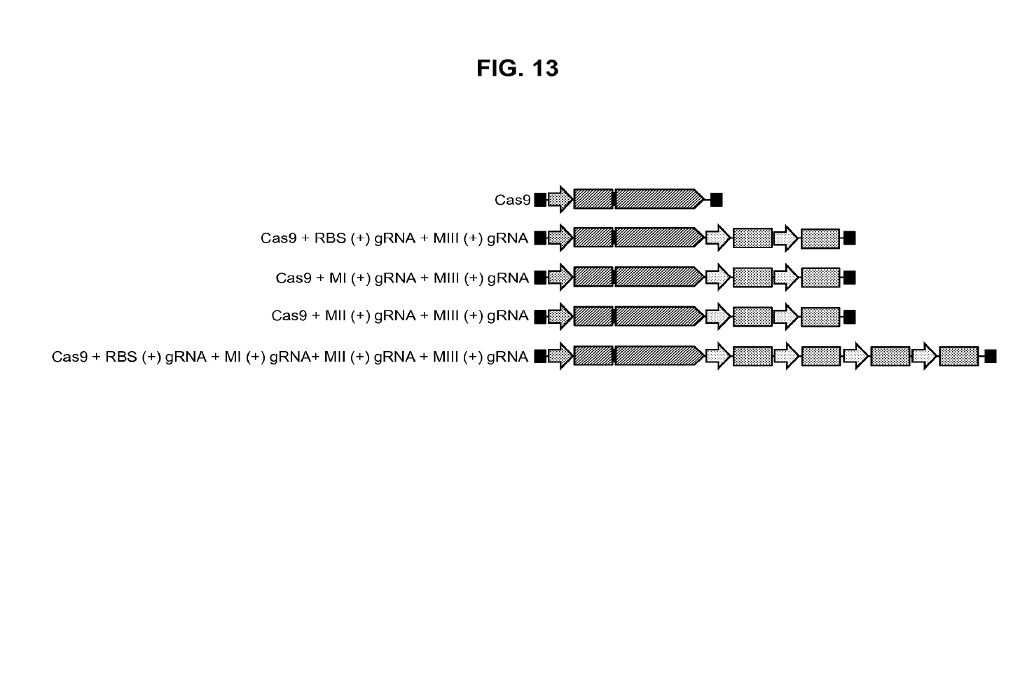

To generate T-DNA vectors containing Cas9 and two gRNAs, the pPAA033 entry vector was modified to contain two gRNAs in tandem. To reduce repetitive sequences within this vector, a second RNA Pol III promoter (At7SL; see, Wang et al., RNA 14:903-913, 2008) was placed upstream of a second gRNA sequence. To enable cloning of sequences within the second gRNA, inverted BsaI sites were positioned within the 5′ region of the gRNA. Further, to facilitate cloning of oligonucleotides into the 5′ region of both gRNAs, a lacZ gene was positioned between the two inverted BsaI sites and a ccdB gene was inserted between the two inverted Esp31 sites. These additions to pPAA033 generated plasmid pAH595, a 2X gRNA entry vector that was used in multisite Gateway recombination reactions along with pCGS710 and pNJB184 to enable construction of the T-DNA plasmids with Cas9 followed by two gRNAs (FIGS. 12 and 13).

To generate T-DNA vectors containing Cas9 and four gRNAs, the gRNA sequences and RNA pol III promoters in pAH595 (after cloning oligonucleotides into the 5′ gRNA regions) were amplified by PCR using primers 5′-TCGTCTCGTCGACCGGC CGCGATGTTGTTGTTACCAG (SEQ ID NO:27) and 5′-GACTCACTATAGGGGAT ATCAGCTGGATGGC (SEQ ID NO:28). The resulting amplicons (containing two gRNA sequences) were digested with SalI and cloned into a different pAH595 vector previously modified to contain desired gRNA sequences. The resulting 4X gRNA vector was used in Gateway recombination reactions along with pCGS710 and pNJB184 to produce T-DNA plasmids with Cas9 followed by four gRNAs (FIGS. 12 and 13).

### Example 2

### Testing CRISPR/Cas Reagents Against Their Target Geminivirus Using Transient Leaf Infiltration Assays

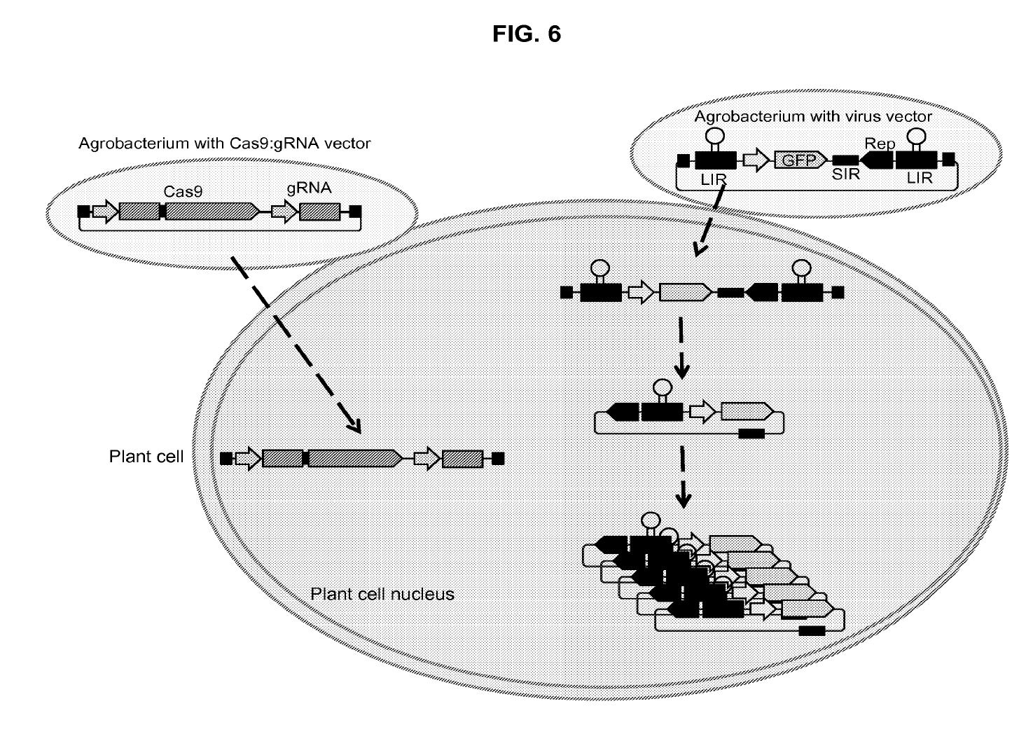

To assess the effectiveness of Cas9 and gRNA(s) against their target geminivirus, a transient assay is employed (FIG. 6). N. benthamiana leaves are infiltrated with a mixture of Agrobacterium strains containing (i) a T-DNA plasmid encoding Cas9 and BeYDV-targeting gRNA(s), and (ii) a T-DNA plasmid harboring 1.2 copies of the BeYDV genome in which the movement and coat protein sequences are replaced with 35S:GFP. When the CRISPR/Cas system is functional against its BeYDV target sequence, GFP expression and replicon copy numbers are reduced relative to a control containing Cas9 and a gRNA with homologous sequence to a different geminivirus. This method is useful for testing any Cas9 and gRNA(s) vector against a target geminivirus, as long as the target geminivirus can be converted into a replicon for delivery of GFP and is amenable to agroinoculation of Nicotiana leaves.

To generate Agrobacterium for infiltration into N. benthamiana leaves, T-DNA plasmids containing the BeYDV genome and CRISPR/Cas components were transformed into Agrobacterium (Agrobacterium tumefaciens GV3101) by the freeze-thaw method (Weigel et al., Cold Spring Harbor Protocols 7:1031-1036, 2005) and plated on Luria-Bertani agar containing 50 μg/mL kanamycin and 50 μg/mL gentamicin. It is to be noted that additional methods for transforming Agrobacterium can be used, including electroporation (Weigel et al., CSH Protocols 7:1-13, 2006). Transformed Agrobacterium colonies were used to inoculate starter cultures containing 5 mL of Luria-Bertani broth with 50 μg/mL kanamycin and 50 μg/mL gentamicin, and the culture was incubated at 28° C. for ˜16 hours. Following the overnight incubation, 100 μL of the starter culture was used to inoculate another culture containing Luria-Bertani broth with 50 μg/mL kanamycin, 50 μg/mL gentamicin, 10 mM 2-(4-morpholino)ethanesulfonic acid (MES; pH 5.6), and 20 μM acetosyringone. Following another overnight incubation at 28° C., cells were pelleted in a centrifuge at 5,000 rpm for 10 minutes. Supernatant was removed and cells were resuspended in infiltration media containing 10 mM MES, 150 μM acetosyringone, and 10 mM MgCl₂. The resulting culture was incubated at room temperature with gentle agitation for two to four hours Immediately before infiltration, Agrobacterium strains were mixed, and the OD₆₀₀ of each strain was adjusted as follows: Agrobacterium containing the T-DNA plasmid with the BeYDV genome was adjusted to a final OD of 0.01; Agrobacterium containing the T-DNA plasmid with CRISPR/Cas reagents was adjusted to a final OD of 0.6.

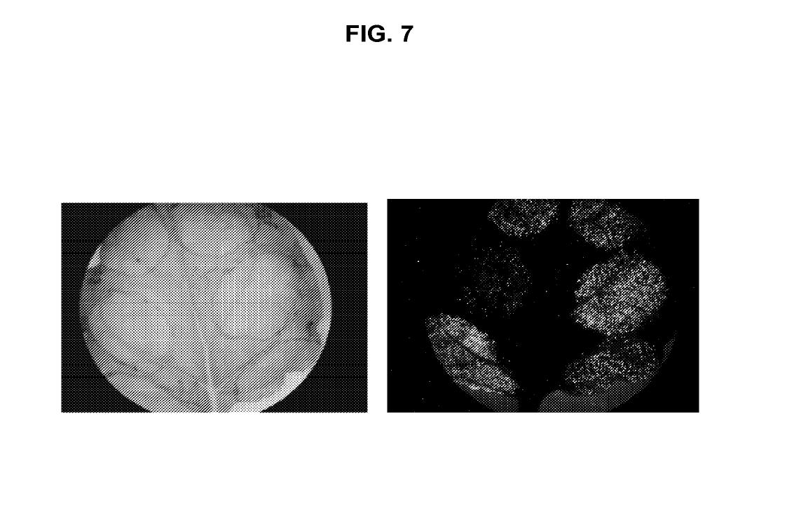

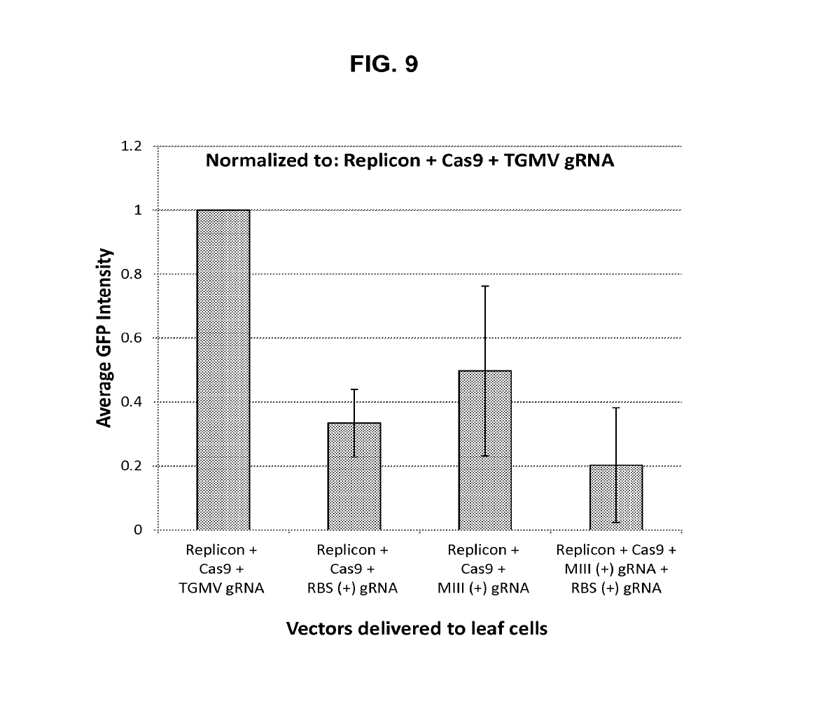

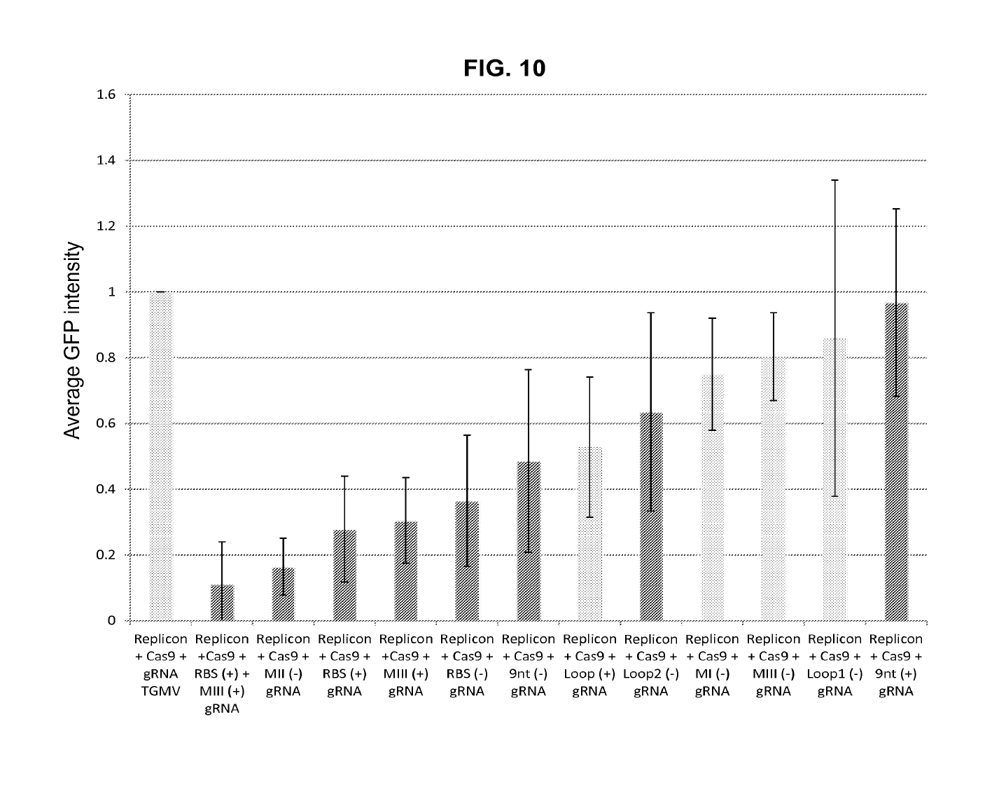

N. benthamiana leaves from plants about 4-6 weeks of age were syringe infiltrated with mixtures of Agrobacterium strains. Each leaf was infiltrated with six to eight different Agrobacterium mixtures. Following infiltration, plastic domes were placed over the plants for 24 hours to maintain high humidity. Five days post infiltration, leaves were removed and photographed (FIG. 7). Quantification of the average GFP intensity within infiltrated leaf tissue was performed using image analysis software (ImageJ). Data points were normalized to an internal control (Replicon+Cas9+TGMV gRNA) and graphed (FIGS. 9 and 10). By normalizing experimental samples to a control within each leaf, variation in GFP-expression was minimized due to properties like leaf age and health. Using this approach, it was observed that most gRNAs were effective at reducing GFP expression from the BeYDV replicon. In addition, some gRNAs were more effective at reducing GFP expression than others. For example, vectors containing a gRNA targeting motif II and complementary to the minus strand reduced GFP expression ˜81%, while vectors containing a gRNA targeting the conserved nonanucleotide sequence and complementary to the plus strand reduced GFP expression only ˜5% (FIG. 10). Differences in GFP expression may be due to differences in CRISPR/Cas activity when targeting different sequences, or due to epigenetic factors at the target site, including DNA secondary structure. For example, and relative to the latter point, vectors containing gRNAs targeting the hairpin within the BeYDV LIR (9nt and loop; FIG. 10) performed, in general, worse than gRNAs targeting the Rep binding site or the motif sequences within the Rep coding sequence, possibly suggesting that DNA secondary structure hinders CRISPR/Cas activity. Taken together, these data demonstrate that CRISPR/Cas systems were effective against target sequences present on geminivirus replicons.

To directly quantify the reduction in viral replication due to the activity of Cas9 and gRNA(s) against their target geminivirus, the transient assay illustrated in FIG. 6 was modified to enable counting of replicon genomes. The T-DNA plasmid containing the BeYDV genome (FIG. 6, upper right) was modified to move the bacterial ColE1 origin of replication from the plasmid backbone into the replicon genome between the GFP and SIR sequences. A β-lactamase gene for ampicillin/carbenicillin resistance was simultaneously introduced at the same location. These changes generated the T-DNA plasmid pAH621. With this configuration, replicational release of the BeYDV genome from the transferred T-DNA in plant cells produced a replicon expressing GFP and carrying sequence necessary for plasmid stability in bacterial cells, including resistance to ampicillin/carbenicillin. Thus, it was predicted that, compared to a replicon-only control, tissue with a replicon exposed to Cas9 and a gRNA targeting the replicon would produce fewer plasmids capable of sustaining bacterial colonies when total DNA extracted from plant leaves was transformed into E. coli. This method is useful for testing any Cas9 and gRNA(s) vector against a target geminivirus, as long as the target geminivirus can be converted into a replicon carrying a bacterial origin of replication and antibiotic resistance gene and is amenable to agroinoculation of plant leaves. It also can be used with a replicon carrying any bacterial origin of replication (e.g., pSC101 or 15A) and any selectable marker gene (e.g., NPTII, tet^{r}, or aadA).

To further quantify the reduction of viral genome replication due to the activity of Cas9 and gRNA(s) against their target geminivirus, the pAH621 T-DNA plasmid carrying the modified BeYDV replicon with the ColE1 origin of replication and β-lactamase gene was syringe-infiltrated as described above for the GFP assays. Five days after infiltration, 0.5 cm leaf punches were isolated from the center of each infiltration point, and total DNA was extracted from the samples with a hexadecyl trimethyl-ammonium bromide (CTAB) extraction buffer. To destroy T-DNA plasmid and replicons released from A. tumefaciens surviving in or on the leaves without eliminating replicons derived from plant cells, the samples were treated with DpnI restriction enzyme, which specifically cleaves Dam methylated DNA originating from prokaryotic cells (Lopez-Ochoa et al., J Virol 80:5841-5853, 2006). Aliquots containing 32 ng of DpnI-treated total DNA were transformed into high efficiency E. coli (“NEB5^{α}”, New England Biolabs catalog #C2987H) according to the manufacturer's protocol, plated on Luria-Bertani agar containing 50 μg/mL carbenicillin, and incubated overnight at 37° C. Plasmids derived by replicational release from the modified replicon in the pAH621 plasmid were distinguished from the parental T-DNA plasmid by sensitivity of the bacterial host to kanamycin and restriction digestion patterns. Fifty randomly selected colonies were tested for sensitivity to kanamycin, and all were susceptible. In addition, restriction enzyme digested plasmid from three randomly selected colonies produced a band pattern consistent with that expected from the replicationally-released modified replicon genome and distinct from the pAH621 parental T-DNA plasmid. Taken together, these data indicate that colonies derived from transformed total DNA extracts consist of plant-derived replicon genomes rather than T-DNA plasmid.

Using this method, colony forming units (CFUs) from total DNA extracted five days after infiltration from leaf spots treated with either replicon only or replicon+Cas9+gRNAs targeting the BeYDV Motif III and the BeYDV RBS were counted (TABLE 2). On average, the presence of the Cas9+gRNAs resulted in an 88% decrease in CFUs relative to tissue treated with the replicon only (p=0.0017). These data indicate that CRISPR/Cas systems reduced geminivirus replicon copy number within plant cells.

### Example 3

### Creating Transgenic Plants that Express CRISPR/Cas Reagents Targeting Geminiviruses

CRISPR/Cas reagents can be integrated into plant genomes for the purpose of reducing disease in whole plants. Due to the ease of multiplexing with CRISPR/Cas, a durable and broad spectrum resistance can be achieved. For example, to create a durable resistance, multiple gRNAs can be designed to facilitate cleavage of different sites within the genome of a single geminivirus. Additionally, to create a broad spectrum resistance, multiple gRNAs can be designed to cleave sequences on more than one geminiviruses (or genetic variants of one type of geminivirus). The above-mentioned features of this technology, together with the ability to transform most plant species, enables nearly any economically-important plant species to be generated with resistance against known geminiviruses.

To demonstrate the effectiveness of this approach, N. benthamiana and tomato plants were transformed with T-DNA vectors containing Cas9 and gRNA(s) that target different geminiviruses. The target geminiviruses for experiments in N. benthamiana, were TGMV and BeYDV. The target virus for tomato was TYLCV. Target sequences for BeYDV were the Rep binding sequence and Rep motif III (TABLE 1), target sequences for TGMV were the Rep binding sequence, Rep motif I, Rep motif II, and Rep motif III, and target sequences for TYLCV were the Rep binding sequence, Rep motif I, Rep motif II, and Rep motif III. Plants were generated that harbor a single T-DNA vector with Cas9 and one or more gRNAs (the latter permits multiplex targeting of a single or multiple geminiviruses; FIGS. 12 and 13).

To integrate plasmid DNA encoding CRISPR/Cas reagents into a plant's genome, any of several methods are used, including Agrobacterium-mediated transformation and biolistic transformation. For examples of Agrobacterium-mediated transformation, see, Horsch et al. Science 227(4691):1229-1231, 1985 (tobacco); Clough et al. The Plant Journal : For Cell and Molecular Biology 16:735-743, 1998 (Arabidopsis); McCormick et al. Plant Cell Reports 5:81-84, 1986 (tomato); and González et al. Plant Cell Reports 17:827-831, 1998 (cassava). For examples of biolistic-mediated transformation, see, Wright et al. Plant Cell Reports 20:429-436, 2001 (maize and wheat). For proof-of-concept experiments, plasmids encoding CRISPR/Cas reagents are integrated into the N. benthamiana and tomato genomes by Agrobacterium-mediated transformation. Following the generation of transgenic plants, Cas9 expression and gRNA expression are assessed by western blot and northern blot, respectively.

### Example 4

### Testing Transgenic Plants for Resistance to Geminiviruses

To determine the extent to which the CRISPR/Cas system confers resistance against geminivirus disease, transgenic plants are first infected with the target geminivirus. Infection is carried out using any of several different methods. These include, for example, biolistic bombardment using plasmids containing partial tandem direct repeats of the geminivirus genomes (see, for example, Muangsan et al., Meth Mol Biol 265:101-115, 2004), agroinfection where the T-DNA molecule contains the partial tandem direct repeats of the geminivirus genome (see, for example, Kheyr-Pour et al., Plant Breeding 112:228-233, 1994), direct inoculation of geminivirus virions using white flies (see, for example, Polston et al., J Vis Exp:JoVE 81, 2013), DNA abrasion (see, for example, Ascencio-Ibanez et al., J Virol Meth 142:198-203, 2007), and transmission of sap from infected plants to a non-infected plant (see, for example, Paplomatas et al., Phytopathol 84:1215-1223, 1994).

Following infection, geminivirus resistance is determined. One method for determining resistance is by visual observation of symptoms (e.g., leaf curling, chlorotic lesions, mosaic, malformation, size reduction, and stunting). Symptoms are scored using a range from 0 to 4, with 0 being no observable symptoms and 4 being severe symptoms (see, for example, Reyes et al., J Virol 87:9691-9706, 2013). Other methods for determining resistance include, for example, quantifying virus copy numbers using techniques such as Southern blotting, leaf disc prints hybridized to a radiolabeled probe, or quantitative polymerase chain reaction (see, for example, Zhang et al., Plant Biotechnol J3:385-397, 2005), as well as enzyme-linked immunosorbent assay (ELISA) and other ELISA-based methods (see, for example, Givord et al., Agronomie 14: 327-333, 1994).

Instead of challenging transgenic plants with a full geminivirus, resistance can be scored by infiltrating leaves from transgenic plants with Agrobacterium containing GFP-expressing geminivirus-replicon vectors (containing 1.2 copies of the geminivirus genome with 35:GFP in replace of the movement and coat protein genes). When delivered to leaf cells on transgenic N. benthamiana plants, geminivirus replicons can amplify and express GFP, but they cannot move from cell to cell. Similar to the transient assays described in Example 2, the activity level of the CRISPR/Cas reagents can be quantified by determining GFP expression and replicon copy numbers about five days post infiltration. Reduced levels of GFP and replicon copy numbers are suggestive of increased geminivirus resistance.

TABLE 1
List of CRISPR/Cas target sequences within geminivirus genomes

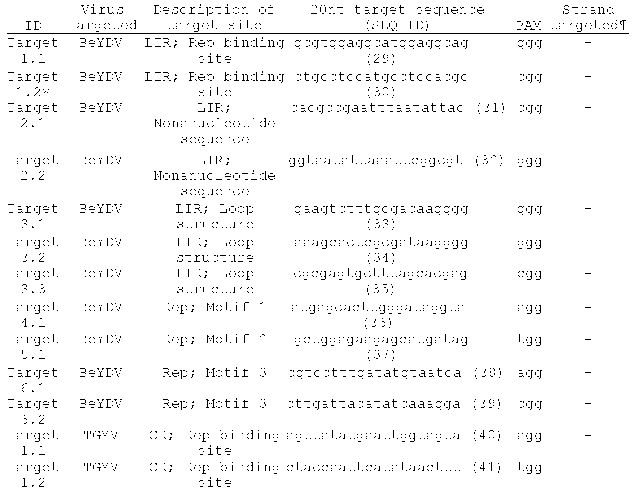

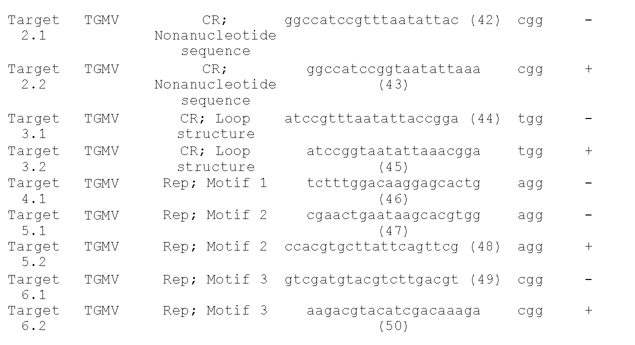

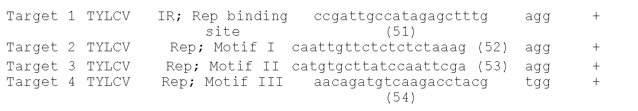

*Shaded cells indicate gRNAs used to generate stable transgenic plants.
¶+ indicates that gRNA is complementary to the plus strand.

TABLE 2
Colony Forming Units (CFUs) observed from transformed total DNA

| | Replicon only | Replicon + Cas9 + | Reduction in CFUs with |
|---|---|---|---|
| Leaf | (CFUs) | gRNAs (CFUs) | Cas9 + gRNAs (%) |
| 1 | 1345 | 106 | 92.1 |
| 2 | 771 | 156 | 79.8 |
| 3 | 1754 | 82 | 95.3 |
| 4 | 1201 | 232 | 80.7 |
| 5 | 1256 | 101 | 92.0 |
| Average | 1265* | 135* | 88.0 |
| SD | 351 | 60 | 7.2 |

*Indicates difference (p = 0.0017) in two-tailed, heteroschedastic t-test.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for generating a plant cell having the potential for increased resistance to geminivirus infection, wherein the method comprises transforming a plant cell with (i) a first nucleic acid containing a sequence that encodes a Clustered Regularly Interspersed Short Palindromic Repeats-associated system (Cas) protein, and (ii) a second nucleic acid containing one or more Clustered Regularly Interspersed Short Palindromic Repeats (CRISPR) RNA (crRNA) sequences and a trans-activating crRNA (tracrRNA) sequence targeted to one or more geminivirus sequences, such that the sequence encoding the Cas protein, the one or more crRNA sequences, and the tracrRNA sequence are stably integrated into the genome of the plant cell.

2. The method of claim 1, further comprising maintaining the plant cell under conditions in which nucleic acids (i) and (ii) are expressed.

3. (canceled)

4. The method of claim 1, wherein the Cas protein is a Cas9 protein with nuclease or nickase activity.

5. (canceled)

6. The method of claim 4, wherein the Cas9 protein is a D10A or H840A nickase.

7. The method of claim 1, wherein the Cas protein is a Cas9 protein without nuclease activity.

8. The method of claim 7, wherein the Cas9 protein is a D10A and H840A protein without nuclease activity.

9-11. (canceled)

12. The method of claim 1, wherein the crRNA and tracrRNA sequences are targeted to a sequence contained within a geminivirus genome.

13-14. (canceled)

15. The method of claim 1, wherein the crRNA and tracrRNA sequences are targeted to DNA beta molecules.

16-17. (canceled)

18. The method of claim 1, wherein each of the one or more crRNA sequences is fused to a tracrRNA sequence.

19. The method of claim 1, wherein the tracrRNA sequence and the one or more crRNA sequences are operably linked to a constitutive promoter.

20-21. (canceled)

22. The method of claim 1, wherein the tracrRNA sequence and the one or more crRNA sequences are operably linked to an inducible promoter.

23. The method of claim 1, wherein the tracrRNA sequence and the one or more crRNA sequences are operably linked to a plant tissue specific promoter.

24. The method of claim 1, wherein the plant cell is in a plant, and wherein the transforming comprises Agrobacterium-mediated transformation, electroporation transformation, polyethylene glycol (PEG) transformation, or biolistic transformation.

25-26. (canceled)

27. The method of claim 1, wherein the second nucleic acid encodes a polycistronic message comprising a tracrRNA sequence and one or more crRNA sequences, or comprising a cr/tracrRNA hybrid (gRNA) sequence.

28. The method of claim 27, further comprising transforming the plant cell with (iii) a third nucleic acid containing a sequence that encodes a protein for processing a transcript expressed from the second nucleic acid.

29-32. (canceled)

33. A plant, plant part, or plant cell that has increased resistance to geminivirus infection, wherein the genome of the plant, plant part, or plant cell comprises a first nucleic acid encoding a Cas protein, and a second nucleic acid containing one or more crRNA sequences and a tracrRNA sequence targeted to one or more geminivirus sequences.

34. (canceled)

35. The plant, plant part, or plant cell of claim 33, wherein the Cas protein is a Cas9 protein with nuclease or nickase activity.

36. (canceled)

37. The plant, plant part, or plant cell of claim 35, wherein the Cas9 protein is a D10A or H840A nickase.

38. The plant, plant part, or plant cell of claim 33, wherein the Cas protein is a Cas9 protein without nuclease activity.

39. The plant, plant part, or plant cell of claim 38, wherein the Cas9 protein is a D10A and H840A protein without nuclease activity.

40. The plant, plant part, or plant cell of claim 33, wherein the sequence encoding the Cas protein is operably linked to a constitutive promoter.

41. The plant, plant part, or plant cell of claim 33, wherein the sequence encoding the Cas protein is operably linked to an inducible promoter.

42. The plant, plant part, or plant cell of claim 33, wherein the sequence encoding the Cas protein is operably linked to a plant tissue specific promoter.

43. The plant, plant part, or plant cell of claim 33, wherein the crRNA and tracrRNA sequences are targeted to a sequence within a geminivirus genome.

44-45. (canceled)

46. The plant, plant part, or plant cell of claim 33, wherein the crRNA and tracrRNA sequences are targeted to DNA beta molecules.

47-48. (canceled)

49. The plant, plant part, or plant cell of claim 33, wherein each of the one or more crRNA sequences is fused to a tracrRNA sequence.

50. The plant, plant part, or plant cell of claim 33, wherein the tracrRNA sequence and the one or more crRNA sequences are operably linked to a constitutive promoter.

51-52. (canceled)

53. The plant, plant part, or plant cell of claim 33, wherein the tracrRNA sequence and the one or more crRNA sequences are operably linked to an inducible promoter.

54. The plant, plant part, or plant cell of claim 33, wherein the tracrRNA sequence and the one or more crRNA sequences are operably linked to a plant tissue specific promoter.

55. The plant, plant part, or plant cell of claim 33, wherein the second nucleic acid encodes a polycistronic message comprising a tracrRNA sequence and one or more crRNA sequences, or comprising a cr/tracrRNA hybrid (gRNA) sequence.

56. The plant, plant part, or plant cell of claim 55, further comprising a third nucleic acid, wherein the third nucleic acid contains a sequence encoding a protein for processing a transcript expressed from the second nucleic acid.

57-60. (canceled)


SEQUENCE LISTING (55 sequences)

SEQ ID NO: 1 - protein, 1379 aa, Streptococcus pyogenese
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 2 - DNA, 13 bp, tomato golden mosaic virus
ggtagtaaggtag

SEQ ID NO: 3 - DNA, 16 bp, mild strain of bean yellow dwarf virus
tggaggcatggaggca

SEQ ID NO: 4 - DNA, 12 bp, mild strain of bean yellow dwarf virus
ttccttacctat

SEQ ID NO: 5 - DNA, 21 bp, mild strain of bean yellow dwarf virus
cactatcatgctcttctccag

SEQ ID NO: 6 - DNA, 21 bp, mild strain of bean yellow dwarf virus
gtccttgattacatatcaaag

SEQ ID NO: 7 - DNA, 15 bp, mild strain of bean yellow dwarf virus
cttcgctgccacgaa

SEQ ID NO: 8 - DNA, 23 bp, mild strain of bean yellow dwarf virus
gcgacaagggggggcccacgccg

SEQ ID NO: 9 - DNA, 26 bp, cabbage leaf curl virus
atgtatcctacaaagtttaggcgtgg

SEQ ID NO: 10 - DNA, 24 bp, cabbage leaf curl virus
ctatgtaattaaacgcatttggag

SEQ ID NO: 11 - DNA, 12 bp, mild strain of bean yellow dwarf virus
ataggtaaggaa

SEQ ID NO: 12 - DNA, 21 bp, mild strain of bean yellow dwarf virus
ctggagaagagcatgatagtg

SEQ ID NO: 13 - DNA, 21 bp, mild strain of bean yellow dwarf virus
ctttgatatgtaatcaaggac

SEQ ID NO: 14 - DNA, 15 bp, mild strain of bean yellow dwarf virus
ttcgtggcagcgaag

SEQ ID NO: 15 - DNA, 20 bp, cabbage leaf curl virus
ctatgtaattaaacgcattt

SEQ ID NO: 16 - DNA, 13 bp, tomato golden mosaic virus
ctaccttactacc

SEQ ID NO: 17 - DNA, 16 bp, mild strain of bean yellow dwarf virus
tgcctccatgcctcca

SEQ ID NO: 18 - DNA, 23 bp, mild strain of bean yellow dwarf virus
cggcgtgggcccccccttgtcgc

SEQ ID NO: 19 - DNA, 26 bp, cabbage leaf curl virus
ccacgcctaaactttgtaggatacat

SEQ ID NO: 20 - DNA, 20 bp, cabbage leaf curl virus
aaatgcgtttaattacatag

SEQ ID NO: 21 - DNA, 21 bp, tomato golden mosaic virus
tttcttacatatcctcagtgc

SEQ ID NO: 22 - DNA, 21 bp, tomato golden mosaic virus
cacctccacgtgcttattcag

SEQ ID NO: 23 - DNA, 21 bp, tomato golden mosaic virus
gtcaagacgtacatcgacaaa

SEQ ID NO: 24 - DNA, 42 bp, Artificial Sequence
  synthetic oligonucleotide
ctccgaattcgcccttcaccatggattacaaggatgatgatg

SEQ ID NO: 25 - DNA, 44 bp, Artificial Sequence
  synthetic oligonucleotide
aaatgtttgaacgatcggacgtctcacttcttcttcttagcctg

SEQ ID NO: 26 - DNA, 104 bp, Artificial Sequence
  synthetic oligonucleotide
gnnnnnnnnnnnnnnnnnnnngttttagagctagaaatagcaagttaaaataaggctagtccgttatcaacttgaaaaagtggcaccgagtcggtgcttttttt

SEQ ID NO: 27 - DNA, 37 bp, Artificial Sequence
  synthetic oligonucleotide
tcgtctcgtcgaccggccgcgatgttgttgttaccag

SEQ ID NO: 28 - DNA, 31 bp, Artificial Sequence
  synthetic oligonucleotide
gactcactataggggatatcagctggatggc

SEQ ID NO: 29 - DNA, 20 bp, bean yellow dwarf virus
gcgtggaggcatggaggcag

SEQ ID NO: 30 - DNA, 20 bp, bean yellow dwarf virus
ctgcctccatgcctccacgc

SEQ ID NO: 31 - DNA, 20 bp, bean yellow dwarf virus
cacgccgaatttaatattac

SEQ ID NO: 32 - DNA, 20 bp, bean yellow dwarf virus
ggtaatattaaattcggcgt

SEQ ID NO: 33 - DNA, 20 bp, bean yellow dwarf virus
gaagtctttgcgacaagggg

SEQ ID NO: 34 - DNA, 20 bp, bean yellow dwarf virus
aaagcactcgcgataagggg

SEQ ID NO: 35 - DNA, 20 bp, bean yellow dwarf virus
cgcgagtgctttagcacgag

SEQ ID NO: 36 - DNA, 20 bp, bean yellow dwarf virus
atgagcacttgggataggta

SEQ ID NO: 37 - DNA, 20 bp, bean yellow dwarf virus
gctggagaagagcatgatag

SEQ ID NO: 38 - DNA, 20 bp, bean yellow dwarf virus
cgtcctttgatatgtaatca

SEQ ID NO: 39 - DNA, 20 bp, bean yellow dwarf virus
cttgattacatatcaaagga

SEQ ID NO: 40 - DNA, 20 bp, tomato golden mosaic virus
agttatatgaattggtagta

SEQ ID NO: 41 - DNA, 20 bp, tomato golden mosaic virus
ctaccaattcatataacttt

SEQ ID NO: 42 - DNA, 20 bp, tomato golden mosaic virus
ggccatccgtttaatattac

SEQ ID NO: 43 - DNA, 20 bp, tomato golden mosaic virus
ggccatccggtaatattaaa

SEQ ID NO: 44 - DNA, 20 bp, tomato golden mosaic virus
atccgtttaatattaccgga

SEQ ID NO: 45 - DNA, 20 bp, tomato golden mosaic virus
atccggtaatattaaacgga

SEQ ID NO: 46 - DNA, 20 bp, tomato golden mosaic virus
tctttggacaaggagcactg

SEQ ID NO: 47 - DNA, 20 bp, tomato golden mosaic virus
cgaactgaataagcacgtgg

SEQ ID NO: 48 - DNA, 20 bp, tomato golden mosaic virus
ccacgtgcttattcagttcg

SEQ ID NO: 49 - DNA, 20 bp, tomato golden mosaic virus
gtcgatgtacgtcttgacgt

SEQ ID NO: 50 - DNA, 20 bp, tomato golden mosaic virus
aagacgtacatcgacaaaga

SEQ ID NO: 51 - DNA, 20 bp, tomato yellow leaf curl virus
ccgattgccatagagctttg

SEQ ID NO: 52 - DNA, 20 bp, tomato yellow leaf curl virus
caattgttctctctctaaag

SEQ ID NO: 53 - DNA, 20 bp, tomato yellow leaf curl virus
catgtgcttatccaattcga

SEQ ID NO: 54 - DNA, 20 bp, tomato yellow leaf curl virus
aacagatgtcaagacctacg

SEQ ID NO: 55 - DNA, 1366 bp, cotton leaf curl virus
accgtgggcgagcggtgtctttggcgttccatgtgggtcccacaatatccaaaagaagaataatggactgggtcaatgcaattgggccttaaatgaaatgggcttggaccagtagattcgagactgggccaatagaataaaacaacaaatggactcataatcaaaacaaagtgtttattcatgtcaaatacattacacactcacacacacacagtcgtacacacatcatattcatcccctatacgtatatcaactaatggggcctcatgcatcatcattatatcaatagcctctaccatgtcctcctggcgaaagtcccgaacatgacaatccctatacatgatctttaatatattatgtatcccttcctccaaattgttgaagtcgaaaggcggtatgatcccatcatggccgtatgggatcatgaaggtcttctttgccagggcaggtgatcttgttgagcacaattcaatccgcacaaggattgaattgtcctcgtggatcttcacgtcgacggtaaataccatccccttctcgttagtatacttgattgtcatttgcatttaattatgaacaacacatgagatgaatcttcttaaatagcgtccatatatttggatatatggacataatgcatatacgtggtgcaataattatcatatgaatatgagtggagacatatatgattgatatctacaggactatcaatcatcgtcaagaaaaaagggataaaagaaaccttattcatgaaaggattaggtagggaaaaataaaagaaggaaattaaggaaaagaaaacccacaatgaaataattaagaaaaaaaaaagaaaaaaagaacacaagaaaggaaaacagaaacatgagcacaaacagaaacctcttgagaaaatattggagcgcagcgggaaaaccaagaaaaacaaaccaaggaagacacttgagcaaaaagggaaaacacaaactaaactaggagggtccaacatgaaattatgaaaagtccgtacacagtaattaatcattaattactgcgcagtaaatgtgaataaaattaacccgaagggttaattttcgagaccccgataggtaattgagtccccaatatatcggggactcaatcggggtcatgagagagaaataaatcccggataccgaaactaccctcaaagctgtgtctggaaggcgcgtgggagtgcgctgaaaaaggtgaccttctctctccaaaaactcaccggaacggccaaactggctgattccggcatctaatcacgacacgcgcggcggtgtgtacccctgggagggtagaccactacgctacgcagcagccttagctacgccggagcttagctcgcccacgttctaatatt